(19) United States
(12) Patent Application Publication
(10) Pub. No.: US 2014/0171704 A1
(43) Pub. Date: Jun. 19, 2014

(54) METHODS AND APPARATUSES FOR PRODUCING ETHYLENE AND PROPYLENE FROM NAPHTHA FEEDSTOCK

(71) Applicant: UOP LLC, Des Plaines, IL (US)

(72) Inventors: Selman Ziya Erisken, Chicago, IL (US); Xin X. Zhu, Long Grove, IL (US)

(73) Assignee: UOP LLC, Des Plaines, IL (US)

(21) Appl. No.: 13/714,125

(22) Filed: Dec. 13, 2012

(51) Int. Cl. C07C 4/04

(52) U.S. Cl. CPC C07C 4/04
USPC 585/303; 422/187; 585/324

(57) ABSTRACT

Method and apparatuses for producing ethylene and propylene from naphtha feedstock are provided. The naphtha feedstock includes a first component consisting of hydrocarbons that have less than or equal to five carbon atoms and a second component. The second component consists of at least one of an isoparaffin component having at least six carbon atoms, a naphthene component having at least six carbon atoms, or an aromatic component having at least six carbon atoms. The naphtha feedstock is separated to produce a first separation stream including the first component and a second separation stream including the second component. At least a portion of the second component from the second separation stream is converted to normal paraffins. Normal paraffins from conversion of the second component and at least a portion of the first component or derivative thereof from the first separation stream are steam cracked to produce ethylene and propylene.

### TECHNICAL FIELD

The technical field generally relates to methods and apparatuses for producing ethylene and propylene from naphtha feedstock, and more particularly relates to methods and apparatuses for producing ethylene and propylene from naphtha feedstock that includes one or more of an aromatic component, a naphthene component, or an isoparaffin component.

### BACKGROUND

Steam cracking is a common industrial process for producing olefins, such as ethylene and propylene, from petroleum-based feedstocks such as naphtha. Steam cracking generally involves pyrolyzing the naphtha in the presence of steam to produce the ethylene and propylene, with other reaction products also being formed during pyrolysis. Ethylene and propylene are widely-used reactants for producing plastics such as polyethylene, polypropylene, and various co-polymers that include ethylene units and propylene units, and the ethylene and propylene are typically separated from the other reaction products that are formed during pyrolysis. Naphtha is generally produced through fractionation of crude oil and/or heavy oil conversion units and has a boiling range of from about 5° C. to about 200° C. Naphtha that is provided for steam cracking is often diverted from gasoline production, although dedicated naphtha streams are also often provided for steam cracking due to the strong commercial demand for ethylene and propylene.

Steam cracking of the naphtha is prone to various inefficiencies. In particular, various normal paraffins in the naphtha can be cracked to ethylene and propylene at relatively high yield and at relatively high conversion rates per pass through a pyrolysis stage. However, many naphtha feedstocks include various components other than normal paraffins that present a wide range of difficulties during steam cracking. For example, typical naphtha feedstocks include aromatic compounds, isoparaffins (i.e., branched paraffins), naphthenes (i.e., cyclic paraffins), as well as the normal paraffins. Steam cracking of aromatic compounds either produces no olefins or produces pyrolysis gas and pyrolysis oil, depending upon the particular type of aromatic compound that are present. Production of pyrolysis oil can lead to fouling within steam cracking apparatuses, thus requiring shutdown for cleaning. Steam cracking of naphthenes generally yields some ethylene and propylene, but also yields significant amounts of aromatic compounds, as well as pyrolysis oil and gases such as hydrogen and methane. Steam cracking of isoparaffins generally produces a mixture of gas and olefins and, thus, is less efficient than steam cracking of normal paraffins. One approach to address the problems caused by the presence of aromatic compounds, naphthenes, and isoparaffins during steam cracking includes use of a high normal paraffin-content naphtha feedstock, which fails to exploit many readily available naphtha feedstocks. Another approach to address the problems caused by the presence of aromatic compounds, naphthenes, and isoparaffins during steam cracking includes or separation of aromatic compounds, napthenes, and/or isoparaffins from the naphtha feedstocks prior to steam cracking. However, separation of the aromatic compounds, naphthenes, and/or isoparaffins from the naphtha feedstocks generally sacrifices yield of some ethylene and propylene from the naphtha feedstock.

Accordingly, it is desirable to provide methods and apparatuses for producing ethylene and propylene from naphtha feedstock that includes one or more of an aromatic component, a naphthene component, or an isoparaffin component with maximized yield of the ethylene and propylene from the naphtha feedstock. Furthermore, other desirable features and characteristics of the present invention will become apparent from the subsequent detailed description of the invention and the appended claims, taken in conjunction with the accompanying drawings and this background of the invention.

### BRIEF SUMMARY

Method and apparatuses for producing ethylene and propylene from naphtha feedstock are provided herein. In an embodiment, a method for producing ethylene and propylene from naphtha feedstock includes providing the naphtha feedstock. The naphtha feedstock includes a first component and a second component. The first component consists of hydrocarbons that have less than or equal to five carbon atoms. The second component consists of at least one of an isoparaffin component having at least six carbon atoms, a naphthene component having at least six carbon atoms, or an aromatic component having at least six carbon atoms. The naphtha feedstock is separated to produce a first separation stream that includes the first component and a second separation stream that includes the second component. At least a portion of the second component from the second separation stream is converted to normal paraffins. Normal paraffins from conversion of the second component and at least a portion of the first component or derivative thereof from the first separation stream are steam cracked to produce ethylene and propylene.

In another embodiment, a method of producing ethylene and propylene from naphtha feedstock is conducted in an apparatus for steam cracking the naphtha feedstock. The method includes providing the naphtha feedstock. The naphtha feedstock includes a first component, a second component, and normal paraffins that have at least six carbon atoms. The first component consists of hydrocarbons that have less than or equal to five carbon atoms. The second component consists of at least one of an isoparaffin component having at least six carbon atoms, a naphthene component having at least six carbon atoms, or an aromatic component having at least six carbon atoms. The naphtha feedstock is separated in a depentanizer unit to produce a first separation stream that includes the first component and a second separation stream that includes the second component. The second separation stream also includes the normal paraffins having at least six carbon atoms. The normal paraffins that have at least six carbon atoms from the second separation stream are adsorbed in a normal paraffin adsorption stage to produce an extract stream and a raffinate stream. The extract stream includes the normal paraffins that have at least six carbon atoms and the raffinate stream includes the second component. At least a portion of the second component from the second separation stream is converted in a conversion stage to produce a first conversion stream that includes normal paraffins that have one or two carbon atoms, a second conversion stream that includes hydrocarbons having three or four carbon atoms, and a third conversion stream that includes conversion products that have at least five carbon atoms. Normal paraffins are steam cracked in a steam cracking stage to produce ethylene and propylene. Normal paraffins that are steam cracked include normal paraffins from at least one of the first conversion stream or the second conversion stream; at least a portion of the first component or derivative thereof from the first separation stream; and the normal paraffins having at least six carbon atoms from the extract stream.

In another embodiment, an apparatus for steam cracking a naphtha feedstock includes a depentanizer unit, a conversion stage, and a steam cracking stage. The depentanizer unit is adapted to receive a naphtha feedstock that includes a first component and a second component, and the depentanizer unit is further adapted to fractionate the naphtha feedstock to produce a first separation stream that includes the first component and a second separation stream. The second separation stream includes the second component, and the second component consists of at least one of an isoparaffin component having at least six carbon atoms, a naphthene component having at least six carbon atoms, or an aromatic component having at least six carbon atoms. The conversion stage is adapted to receive at least a portion of the second component from the second separation stream. The conversion stage is further adapted to convert at least a portion of the second component from the second separation stream to normal paraffins. The steam cracking stage is adapted to receive and steam crack normal paraffins from the conversion stage and at least a portion of the first component or derivative thereof from the depentanizer unit to produce ethylene and propylene.

### BRIEF DESCRIPTION OF THE DRAWINGS

The various embodiments will hereinafter be described in conjunction with the following drawing figures, wherein like numerals denote like elements, and

FIG. 1 is a schematic diagram of an apparatus and method for producing ethylene and propylene from naphtha feedstock in accordance with an exemplary embodiment; and

FIG. 2 is a schematic diagram of an apparatus and method for producing ethylene and propylene from naphtha feedstock in accordance with another exemplary embodiment.

### DETAILED DESCRIPTION

The following detailed description is merely exemplary in nature and is not intended to limit the various embodiments or the application and uses thereof. Furthermore, there is no intention to be bound by any theory presented in the preceding background or the following detailed description.

Methods and apparatuses for producing ethylene and propylene from naphtha feedstock are provided herein. The methods and apparatuses enable use of full boiling range naphtha (FBR) feedstocks for production of ethylene and propylene through steam cracking with maximized yield of ethylene and propylene from the naphtha feedstock. The maximized yield of ethylene and propylene is achieved by separating normal paraffin fractions at various stages within the apparatus for steam cracking. Certain non-normal paraffin components of the naphtha feedstock may then be converted to normal paraffins, followed by separation of the normal paraffins from other conversion products, to increase the yield of normal paraffins from the naphtha feedstock. In this manner, unnecessary exposure of normal paraffins that produce high ethylene and paraffin yield through steam cracking may be avoided while effectively recovering normal paraffins from other components in the naphtha feedstock that produce lesser yields of ethylene and propylene through steam cracking than normal paraffins. As a result, maximized yield of normal paraffins from the naphtha feedstock is achieved regardless of the initial composition of the naphtha feedstock. The normal paraffins may then be steam cracked to maximize yield of ethylene and propylene from the naphtha feedstock.

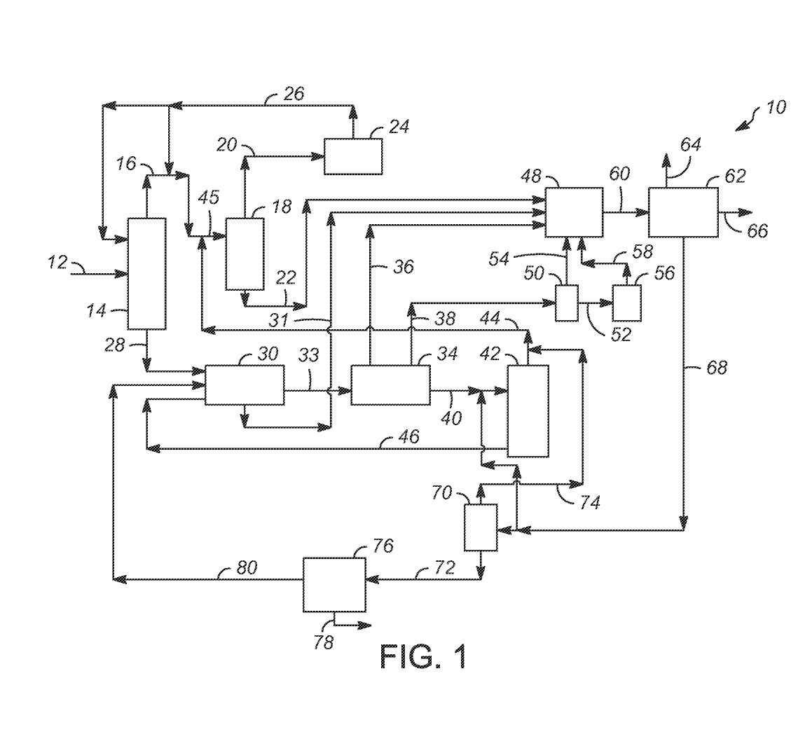

An embodiment of a method and an apparatus 10 for producing ethylene and propylene from naphtha feedstock 12 will now be described with reference to FIG. 1. In accordance with an exemplary method, the naphtha feedstock 12 is provided. As alluded to above, FBR naphtha feedstocks 12 may be used in accordance with the methods and apparatuses described herein, although it is to be appreciated that the methods and apparatuses that are described herein are not limited to use of FBR naphtha feedstocks 12. Suitable naphtha feedstock 12 that may be used, for purposes of the methods and apparatuses described herein, include any naphtha feedstock 12 that includes a first component of hydrocarbons that have less than or equal to five carbon atoms and a second component of at least one of an isoparaffin component having at least six carbon atoms, a naphthene component having at least six carbon atoms, or an aromatic component having at least six carbon atoms. In particular, the second component may include any combination of one or more of the isoparaffin component having at least six carbon atoms, the naphthene component having at least six carbon atoms, or the aromatic component having at least six carbon atoms. Whereas the naphtha feedstock 12 may include additional components beyond the first component and the second component, the “first component” and the “second component”, as referred to herein, only encompass the above-referenced compounds and the designation of “first component” and “second component” is employed for simplicity of description. In an embodiment, the first component specifically includes isopentane and/or normal pentane. Further, in an embodiment, the naphtha feedstock 12 also includes normal paraffins that have at least six carbon atoms, such as normal paraffins that have from six to twelve carbon atoms. FBR naphtha feedstocks 12, as referred to herein, include hydrocarbon feedstocks that primarily include hydrocarbons having from five to twelve carbon atoms, e.g., at least 90 weight % of C5 to C12 hydrocarbons based upon the total weight of the naphtha feedstock 12, and the FBR naphtha feedstocks 12 include at least the first component and the second component as described above.

The naphtha feedstock 12 is separated to produce a first separation stream 16 including the first component and a second separation stream 28 including the second component. In an embodiment and as shown in FIG. 1, the apparatus 10 includes a depentanizer unit 14 that is adapted to receive the naphtha feedstock 12 and that is further adapted to fractionate the naphtha feedstock 12 to produce the first separation stream 16 and the second separation stream 28. Depentanizer units are known in the art and, although not shown in the Figures, generally include a depentanizer column, an overhead receiver with optional reflux pipes that lead back to the depentanizer column, and a heater. The depentanizer unit 14 fractionates the naphtha feedstock 12 to produce the first separation stream 16 as a fractionation overhead, with the first separation stream 16 including the first component as described above as well as any other compounds that are present in the naphtha feedstock 12 and that have a higher vapor pressure than pentane. Although not shown, it is to be appreciated that the apparatus 10 may include one or more upstream separation stages that are used to separate compounds from the naphtha feedstock 12 that have higher vapor pressures than pentane such that the first separation stream 16 includes substantially only pentanes, e.g., pentanes in an amount of at least 95 weight %, such as from 98 weight % to about 100 weight %, based on the total weight of the first separation stream 16. In an embodiment, the naphtha feedstock 12 also includes the normal paraffins that have at least six carbon atoms, such as normal paraffins that have from six to twelve carbon atoms. The normal paraffins that have at least six carbon atoms are separated in the second separation stream 28 along with the second component.

In an embodiment and as shown in FIG. 1, the first component includes normal pentane and the normal pentane is separated from the first separation stream 16 to form a normal pentane-containing stream 22, which may then be provided for steam cracking as described in further detail below. In particular, as shown in FIG. 1, the apparatus 10 further includes a deisopentanizer unit 18 that is adapted for receiving the first separation stream 16 and that is further adapted to separate the first separation stream 16 into the normal pentane-containing stream 22 and an isopentane-containing stream 20. Deisopentanizer units are known in the art. In this embodiment, at least a portion of the isopentane from the first separation stream 16 and, more specifically, from the isopentane-containing stream 20 is converted to normal paraffins. For example, the isopentane-containing stream 20 may be provided to a isopentane reverse isomerization unit 24, where at least a portion of the isopentane from the first separation stream 16 is reverse isomerized in the presence of an isopentane reverse isomerization catalyst to produce normal paraffins and, optionally, other reaction products in an isopentane conversion product stream 26. Reverse isomerization of the isopentane-containing stream 20, as referred to herein, is an isomerization technique that employs an isopentane reverse isomerization catalyst and isomerization conditions that result in conversion of at least some isopentane in the isopentane-containing stream 20 to normal paraffins. Isopentane reverse isomerization units, as well as isopentane reverse isomerization catalysts and conditions for conducting reverse isomerization of isopentane, are known to those of skill in the art. Examples of suitable isopentane reverse isomerization catalysts include, but are not limited to, zeolitic catalysts and sulfated zirconia catalysts. Exemplary conditions for conducting reverse isomerization of isopentane include a temperature of from about 135 to about 700° C. and a pressure of from about 1378 to about 3103 kPa. Depending upon the particular type of isopentane reverse isomerization catalyst that is used, as well as particular reverse isomerization conditions, various different hydrocarbons are produced in addition to normal paraffins. For example, the isopentane conversion product stream 26 may include hydrocarbons that have at least six carbon atoms, in which case at least a portion of the isopentane conversion product stream 26 may be returned to the depentanizer unit 14 as shown in FIG. 1. Further, the isopentane conversion product stream 26 may include hydrocarbons that have four carbon atoms, in which case at least a portion of the isopentane conversion product stream 26 may be returned to a debutanizer unit (not shown). As also shown in FIG. 1, at least a portion of the isopentane conversion product stream 26 may be combined with the first separation stream 16 prior to introduction into the deisopentanizer unit 18, such as under circumstances where substantially no hydrocarbons having greater than five carbon atoms are included in the isopentane conversion product stream 26. In this embodiment, normal pentane that is derived from the conversion of isopentane may be separated along with the normal pentane from the naphtha feedstock 12 and included in the normal pentane-containing stream 22. Thus, when the first component includes isopentane and the isopentane is converted to normal paraffins, such normal pentane is considered a derivative of the first component, and the normal pentane-containing stream 22 may include the normal pentane that is the derivative of the first component.

In an embodiment and as shown in FIG. 1, the second separation stream 28 includes the normal paraffins that have at least six carbon atoms from the naphtha feedstock 12, in addition to the second component. In this embodiment and as shown in FIG. 1, the apparatus 10 further includes a normal paraffin adsorption stage 30 that includes an adsorbent (not shown) that selectively adsorbs normal paraffins over other hydrocarbons such as aromatic compounds, naphthenes, and isoparaffins to produce an extract stream 31 and a raffinate stream 33. The extract stream 31 includes the normal paraffins that have at least six carbon atoms, and the raffinate stream 33 includes the second component (as well as any hydrocarbons other than normal paraffins and the above-mentioned compounds of the second component that may be present in the second separation stream 28). The adsorbent may further selectively adsorb normal paraffins that have six or more carbon atoms, such as from six to twelve carbon atoms, over other normal paraffins such as those that have five or less carbon atoms. Thus, the normal paraffin adsorption stage 30 desirably receives the second separation stream 28, which generally only includes hydrocarbons that have at least six carbon atoms. The normal paraffin adsorption stage 30 is desirably disposed downstream of the depentanizer unit 14 because adsorption separation of normal paraffins that have less than or equal to five carbon atoms from the second component is often inefficient or ineffective. Normal paraffin adsorption stages, as well as adsorbents that selectively adsorb normal paraffins, are known in the art, as are adsorbents that particularly adsorb normal paraffins that have at least six carbon atoms over other normal paraffins that have less than six carbon atoms. It is to be appreciated that the apparatus 10 may include the normal paraffin adsorption stage 30 even when the second separation stream 28 is free from normal paraffins that have at least six carbon atoms. For example, a C6 hydrocarbon stream 80 that includes normal paraffins having at least six carbon atoms in embodiments, such as an aromatic-depleted conversion raffinate stream 80, may be provided to the normal paraffin adsorption stage 30 for separation of the normal paraffins that have at least six carbon atoms therefrom. The normal paraffins that have at least six carbon atoms from the extract stream 31 may be provided for steam cracking as described in further detail below, while the raffinate stream 33 that includes the second component is subject to further processing as described below.

In accordance with the exemplary method, at least a portion of the second component from the second separation stream 28 is converted to normal paraffins, thereby maximizing the yield of normal paraffins from the naphtha feedstock 12. In particular, at least one of an isoparaffin component having at least six carbon atoms, a naphthene component having at least six carbon atoms, or an aromatic component having at least six carbon atoms from the second separation stream 28 and, more particularly from the raffinate stream 33, is converted to normal paraffins for downstream steam cracking. In this regard, the exemplary apparatus 10 of FIG. 1 includes a conversion stage 34 that is adapted to receive at least a portion of the second component from the second separation stream 28, and the conversion unit is further adapted to convert at least a portion of the second component from the second separation stream 28 to normal paraffins. In embodiments, substantially all of the second component from the second separation stream 28, e.g., at least 99 weight % of the second component based on the total weight of the second separation stream 28, is provided to the conversion unit. In embodiments, a substantial portion of the second component from the naphtha feedstock 12 is provided to the conversion unit, such as at least 50 weight %, or such as from 90 to about 100 weight %, of the second component based on the total weight of the naphtha feedstock 12.

Conversion techniques for converting at least the portion of the second component to normal paraffins can vary based upon the chemical makeup of the second component, and any conversion technique is suitable that is effective to convert at least a portion of the second component to normal paraffins. Conversion techniques for converting isoparaffin components having at least six carbon atoms, naphthene components having at least six carbon atoms, and aromatic components having at least six carbon atoms to normal paraffins are known in the art. In various embodiments and as described in further detail below, reverse isomerization and catalytic reforming are examples of two conversion techniques that are suitable for converting the second component to normal paraffins, and such techniques are described in further detail below in the context of specific embodiments.

Conversion of at least the portion of the second component to normal paraffins generally results in a range of conversion products, regarding of particular conversion techniques that are employed to convert the second component to normal paraffins, and the normal paraffins are generally only one type of conversion product that is produced. For example, normal paraffins having from one to ten carbon atoms may be produced through various conversion techniques from the second component, and other compounds such as isoparaffins and aromatic compounds may also be produced depending upon the particular conversion technique that is employed. Within the conversion unit, the conversion products are generally separated through known separation techniques including fractionation, adsorption, and the like. In particular, although not shown in the Figures, the conversion unit may include a reactor for converting at least a portion of the second component to various conversion products, and the conversion unit may further include various separation units for separating the conversion products. In an embodiment and as shown in FIG. 1, at least the portion of the second component from the second separation stream 28 is converted to produce a first conversion stream 36 that includes normal paraffins having one or two carbon atoms, a second conversion stream 38 that includes hydrocarbons having three or four carbon atoms, and a third conversion stream 40 that includes conversion products having at least five carbon atoms.

In an embodiment, the first conversion stream 36 includes ethane, and optionally includes methane, and the first conversion stream 36 is provided for steam cracking as described in further detail below. In this regard, the first conversion stream 36 may be conveyed from the conversion unit in vapor form. The first conversion stream 36 may be provided for steam cracking in the absence of further separation of compounds therefrom; however, it is to be appreciated that in embodiments (not shown), methane can be separated from the first conversion stream 36. The second conversion stream 38 may also be provided for steam cracking, as described in further detail below, along with the first conversion stream 36, in the absence of further separation of compounds therefrom. However, in an embodiment, the second conversion stream 38 includes propane, normal butane, and/or isobutane, and the isobutane may be separated for conversion to normal paraffins. In particular, in this embodiment, the second conversion stream 38 may include propane, normal butane, or isobutane, or any combination of propane, normal butane, and isobutane. The second conversion stream 38 is generally referred to in the art as a liquefied petroleum gas (LPG) stream. In this embodiment, the propane and normal butane may be separated from the isobutane to maximize yield of normal paraffins. Although not shown in the Figures, propane may be separated from hydrocarbons having four carbon atoms in the second conversion stream 38.

As shown in FIG. 1, the second conversion stream 38, more particular a propane fraction and/or a C4 hydrocarbon fraction from the second conversion stream 38, is provided to an isoparaffin separation stage 50. Although not shown in the Figures, the isoparaffin separation stage 50 may include a depropanizer unit and a deisobutanizer unit for separating the propane and C4 hydrocarbon fractions into a propane stream (not shown), a normal butane stream (not shown), and an isobutane stream. Alternatively and as shown in FIG. 1, propane from the second conversion stream 38 may be separated along with normal butane in a combined C3/C4 stream 54, with isobutane stream 52 including isobutane. Although FIG. 1 shows a single stream 54 that includes propane and normal butane, it is to be appreciated that separate streams can be provided including propane or normal butane. The combined C3/C4 stream 54 may be provided for steam cracking, and isobutane in the isobutane stream 52 may be further converted to normal paraffins. In particular, at least a portion of the isobutane from the second conversion stream 38 is converted to normal paraffins, such as in an isobutane conversion zone 56. The isobutane conversion zone 56 may include one or more isobutane conversion units (not shown) for producing normal paraffins from isobutane. For example, in an embodiment, the isobutane stream 52 is provided to a reverse butamer unit, where at least a portion of the isobutane is converted to normal paraffins in the presence of an isobutane reverse isomerization catalyst. Reverse butamer units, as well as isobutane reverse isomerization catalysts and conditions for conducting reverse isomerization of isobutane, are known in the art. The normal paraffins produced from conversion of the isobutane stream 52 are included in a C4 conversion stream 58 and may be provided for steam cracking as described in further detail below. Although not shown, it is to be appreciated that the C4 conversion stream 58 may be further separated, such as by returning the C4 conversion stream 58 to the isoparaffin separation stage 50.

As set forth above, in an embodiment, the third conversion stream 40 includes conversion products having five carbon atoms and conversion products having at least six carbon atoms. More specifically, the third conversion stream 40 includes any products of conversion in the conversion stage 34 that have five or more carbon atoms, and no products of the third conversion stream 40 are directly provided for steam cracking. In an embodiment and as shown in FIG. 1, the third conversion stream 40 is separated to produce a separated conversion stream 46 that includes conversion products that have at least six carbon atoms and a recycle stream 44 that includes conversion products that have five carbon atoms. For example, the apparatus 10 may include a second depentanizer unit 42 that is adapted to receive the third conversion stream 40, with the third conversion stream 40 separated in the second depentanizer unit 42 through conventional techniques to produce the separated conversion stream 46 and the recycle stream 44. In an embodiment, the recycle stream 44 and the first separation stream 16 are combined to form a combined pentane stream 45 that is upstream of the deisopentanizer unit 18. In this embodiment, the first component from the naphtha feedstock 12 and the recycle stream 44 may both include isopentane and normal pentane, although it is to be appreciated that the first component and the recycle stream 44 may include only one of isopentane or normal pentane provided that the resulting combined pentane stream 45 includes both isopentane and normal pentane to necessitate separation in the deisopentanizer unit 18. The isopentane is then separated from the normal pentane in the combined pentane stream 45 in the manner described above. Thus, in this embodiment, the normal pentane-containing stream 22 further includes normal pentane from the recycle stream 44, or normal pentane that is derived from isopentane in the recycle stream 44.

As set forth above, the separated conversion stream 46 includes conversion products that have at least six carbon atoms, and the separated conversion stream 46 may be produced through separation of the third conversion stream 40 in the second depentanizer unit 42. Further processing of the separated conversion stream 46 is dependent upon a chemical makeup of the third conversion stream 40, and the chemical makeup of the third conversion stream 40 varies depending upon the particular type of conversion technique that is employed for converting the second component to normal paraffins. In an embodiment at least the portion of the second component from the second separation stream 28 is converted to normal paraffins through reverse isomerization in the presence of a reverse isomerization catalyst to produce normal paraffins. In this embodiment and as shown in FIG. 1, the conversion unit is a reverse isomerization unit. Reverse isomerization of at least the portion of the second component, as referred to herein, is similar to reverse isomerization as described above in the context of reverse isomerizing the isopentane. In particular, during reverse isomerization of at least the portion of the second component, the reverse isomerization catalyst is employed under isomerization conditions that result in conversion of at least some of the second component to normal paraffins. Reverse isomerization units for reverse isomerizing hydrocarbon compounds having at least six carbon atoms, as well as reverse isomerization catalysts and conditions for conducting reverse isomerization of hydrocarbon compounds having at least six carbon atoms, are known to those of skill in the art. The reverse isomerization catalyst can be the same as or different from the isopentane reverse isomerization catalyst that is described above. Specific examples of suitable reverse isomerization catalysts for reverse isomerizing hydrocarbons that have at least six carbon atoms include, but are not limited to, zeolitic, sulfonated zirconia, and chlorided alumina catalysts. Exemplary conditions for conducting reverse isomerization of the second component include a temperature of at most 371° C. Without being bound to any particular theory, it is believed that any paraffins and isoparaffins that are present during reverse isomerization will crack to paraffins that have less carbon atoms, and aromatic compounds and naphthenes will go through ring opening to produce paraffins. Also without being bound to any particular, theory, it is believed that reaction temperatures below 371° C. will minimize conversion of naphthenes to aromatic compounds.

During reverse isomerization of at least the portion of the second component, isoparaffins are converted to normal paraffins, and naphthenes and aromatic compounds of the second component are subject to ring-opening to produce normal paraffins and/or isoparaffins. For example, reverse isomerizing at least the portion of the second component from the second separation stream 28 generally produces normal paraffins and isoparaffins that have from one to greater than five carbon atoms. More specifically, methane, as well as hydrocarbons that have from two to four carbon atoms are produced and may be provided in the first conversion stream 36 and the second conversion stream 38 as described above. Additionally, normal paraffins and isoparaffins that have at least five carbon atoms are produced and are provided in the third conversion stream 40, which is separated as described above to produce the recycle stream 44 and the separated conversion stream 46. In this embodiment, because conversion is conducted through reverse isomerization, aromatic compounds and naphthenes are consumed during conversion.

Because aromatic compounds and napthenes are not produced and, rather, are converted to normal paraffins and isoparaffins during reverse isomerization, a separate aromatic separation stage is not necessary. In an embodiment and as shown in FIG. 1, the normal paraffins and isoparaffins that have at least six carbon atoms from the separated conversion stream 46 may be again reverse isomerized, such as by returning the separated conversion stream 46 to the normal paraffin adsorption stage 30. Alternatively, although not shown, the separated conversion stream 46 may be provided directly to the conversion stage 34.

Normal paraffins from conversion of the second component and at least a portion of the first component or derivative thereof from the first separation stream 16 are steam cracked to produce ethylene and propylene. A “derivative” of the first component includes, for example, normal paraffins that are produced through conversion of any isopentane in the first component as described above. In particular, normal pentane from the normal pentane-containing stream 22 and any combination of the normal paraffins from the first conversion stream 36 (e.g., methane and ethane) and/or from the second conversion stream 38 (e.g., propane and normal butane), optionally with normal paraffins from the extract stream 31 (e.g., normal paraffins having at least six carbon atoms), may be steam cracked together. Maximized yield of ethylene and propylene is achieved due to separation and conversion of the second component to normal paraffins instead of providing the second component for steam cracking. In particular, isoparaffins that have at least six carbon atoms, naphthenes that have at least six carbon atoms, and aromatic compounds that have at least six carbon atoms all reduce yield of ethylene and propylene from the naphtha feedstock 12, and the methods and apparatuses described herein minimize any amounts of the second component that are present during steam cracking while maximizing amounts of normal paraffins that are provided for steam cracking. Referring to FIG. 1, steam cracking may be conducted in a steam cracking stage 48, where the normal paraffins are pyrolyzed in the presence of steam to produce ethylene, propylene, and various hydrocarbon by-products. Steam cracking stages, as well as conditions for conducting steam cracking, are known in the art. In an embodiment, steam cracking the normal paraffins produces a cracked product stream 60 that includes ethylene, propylene, and steam-cracked hydrocarbons that have at least five carbon atoms as by-products, with gases such as hydrogen and methane also produced. In this embodiment and as shown in FIG. 1, the cracked product stream 60 is separated in a cracked product separation unit 62, which may include various separation stages (not shown) for producing a pyrolysis gas stream 64 that includes, for example, hydrogen and methane; an ethylene/propylene product stream 66 that includes the ethylene and propylene; and a pyrolysis gasoline stream 68 that includes steam-cracked hydrocarbons that have from five to twelve carbon atoms (e.g., isoparaffins, aromatic compounds, and the like). Although not shown, a pyrolysis oil stream that has hydrocarbons having greater than 12 carbon atoms may also be separated after steam cracking. Although also not shown, the pyrolysis gasoline stream 68 may be hydrotreated to effectuate olefin saturation. In an embodiment and as shown in FIG. 1, at least a portion of the steam-cracked hydrocarbons that have from five to twelve carbon atoms are recovered for converting to normal paraffins with at least the portion of the second component from the second separation stream 28. In particular, in an embodiment and as shown in FIG. 1, the pyrolysis gasoline stream 68 is provided to the second depentanizer unit 42 and/or a third depentanizer unit 70, where the pyrolysis gasoline stream 68 is separated to recover hydrocarbons that have five carbon atoms from other hydrocarbons that have at least six carbon atoms. For example, when provided to the third depentanizer unit 70, the pyrolysis gasoline stream 68 may be separated into a recovered pentane stream 74 and a separated aromatic-containing stream 72. The recovered pentane stream 74 may include normal pentane and/or isopentane, and the recovered pentane stream 74 may be combined with the recycle stream 44 and the separated aromatic-containing stream 72. The separated aromatic-containing stream 72 may include aromatic compounds and hydrocarbons that have at least six carbon atoms that are produced from steam cracking, and the separated aromatic-containing stream 72 may be further separated in an aromatic separation stage 76 to produce an aromatic stream 78 and an aromatic-depleted conversion raffinate stream 80 that includes the hydrocarbons that have at least six carbon atoms that are produced from steam cracking. In an embodiment and as shown in FIG. 1, hydrocarbons from having at least six carbon atoms from the aromatic-depleted conversion raffinate stream 80 may be again reverse isomerized, such as by returning the aromatic-depleted conversion raffinate stream 80 to the normal paraffin adsorption stage 30. Alternatively, although not shown, the aromatic-depleted conversion raffinate stream 80 may be provided directly to the conversion stage 34.

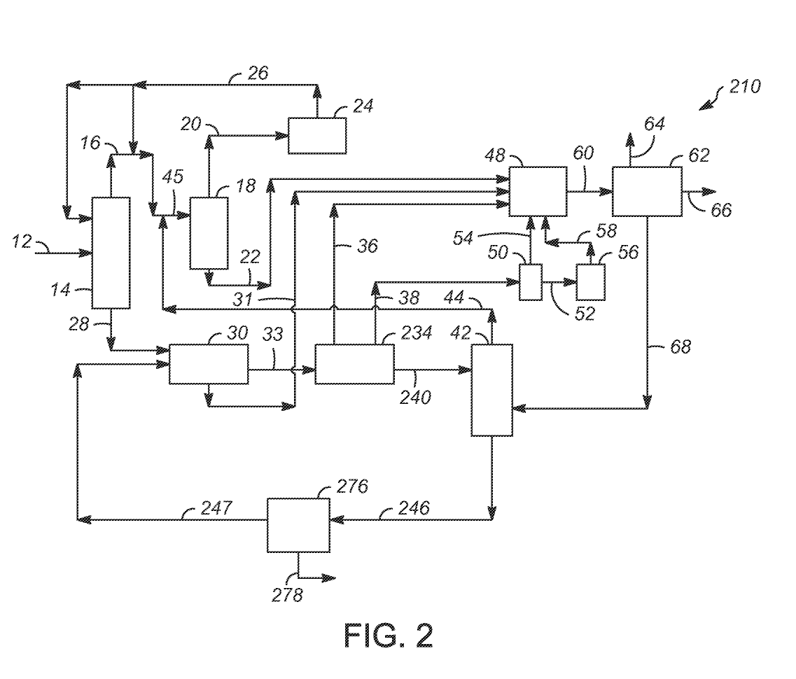

Another embodiment of a method and apparatus 210 for producing ethylene and propylene from naphtha feedstock 12 will now be described with reference to FIG. 2. In this embodiment, the apparatus 210 and method is the same as the apparatus 10 and method described above in the context of FIG. 1 up to the conversion stage, and the same description above for suitable naphtha feedstocks equally applies to the embodiment of the apparatus 10 and method shown in FIG. 2. In the embodiment of FIG. 2, the second component from the second separation stream 28 is converted to normal paraffins through catalytic reforming. In particular, the conversion stage 234 of the apparatus 210 is a catalytic reforming stage 234 that catalytically reforms at least the portion of the second component from the second separation stream 28 in the presence of a platinum- and/or rhenium-containing catalyst to produce normal paraffins and an aromatic conversion component, among other conversion products as known in the art. Catalytic reforming in the presence of the platinum- and/or rhenium-containing catalyst is generally known in the art as platforming. The first conversion stream 36 and the second conversion stream 38 are produced in the same manner as described above and have the same chemical makeup as described above. However, unlike reverse isomerization, catalytic reforming also produces aromatic compounds, referred to collectively herein as “the aromatic conversion component”. In this embodiment, the aromatic conversion component is included in the third conversion stream 240 separate from the first conversion stream 36 and the second conversion stream 38. In particular, the third conversion stream 240 specifically includes conversion products that have at least five carbon atoms, and the third conversion stream 240 also includes the aromatic conversion component.

The third conversion stream 240 is separated to produce a separated conversion stream 246 and a recycle stream 44 in the same manner as described above. However, due to conversion being conducted through catalytic reforming and the third conversion stream 240 including the aromatic component, a chemical makeup of the separated conversion stream 246 is different than the separated conversion stream 246 described above for the embodiment of the method and apparatus 10 shown in FIG. 1. In particular, the separated conversion stream 246 includes the aromatic conversion component and any other conversion products that have at least six carbon atoms. Because aromatic compounds are produced through catalytic reforming in this embodiment and are not consumed, aromatic compounds are removed from the apparatus 210 as a separate product or intermediate stream for other downstream unit operations. As such, in this embodiment and as shown in FIG. 2, the separated conversion stream 246 is further separated in an aromatic separation stage 276 to produce an aromatic stream 278 and an aromatic-depleted raffinate stream 247. The aromatic stream 278 includes the aromatic conversion component, and the aromatic-depleted raffinate stream 247 includes any other conversion products that have at least six carbon atoms, other than aromatic compounds. As shown in FIG. 2, the conversion products that have at least six carbons atoms, from the aromatic-depleted raffinate stream 247, may be again catalytically reformed such as by returning the aromatic-depleted raffinate stream 247 to the normal paraffin adsorption stage 30 to separate any normal paraffins that have at least six carbon atoms from the aromatic-depleted raffinate stream 247. Alternatively, although not shown, the aromatic-depleted raffinate stream 247 may be provided directly to the conversion stage 234.

In an embodiment and as shown in FIG. 2, because the third conversion stream 240 includes the aromatic conversion component, the pyrolysis gasoline stream 68 is provided to the second depentanizer unit 42 to be separated along with the third conversion stream 240. In this embodiment, the third depentanizer unit is not necessary because aromatic contamination of the separated conversion stream 46 is not a concern as it may be in the embodiment shown in FIG. 1. In the embodiment shown in FIG. 2, the pyrolysis gasoline stream 68 is separated to recover hydrocarbons that have five carbon atoms from other hydrocarbons that have at least six carbon atoms. The hydrocarbons that have five carbon atoms from the pyrolysis gasoline stream 68 are included in the recycle stream 44, as described in detail above, and the other hydrocarbons that have at least six carbon atoms from the pyrolysis gasoline stream 68 are included in the separated conversion stream 246 along with the aromatic conversion component and any other conversion products that have at least six carbon atoms. The separated conversion stream 246 is further separated to produce the aromatic stream 278 and an aromatic-depleted raffinate stream 247 as described above.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A method of producing ethylene and propylene from naphtha feedstock, the method comprising:
providing the naphtha feedstock comprising:
a first component consisting of hydrocarbons having less than or equal to five carbon atoms; and
a second component consisting of at least one of an isoparaffin component having at least six carbon atoms, a naphthene component having at least six carbon atoms, or an aromatic component having at least six carbon atoms;
separating the naphtha feedstock to produce a first separation stream including the first component and a second separation stream including the second component;
converting by single stage reverse isomerization in the presence of a reverse isomerization catalyst at least a portion of the second component from the second separation stream to normal paraffins; and
steam cracking normal paraffins from conversion of the second component and at least a portion of the first component or derivative thereof from the first separation stream to produce ethylene and propylene.

2. The method of claim 1, wherein the first component comprises normal pentane, and wherein steam cracking the normal paraffins from conversion of the second component and at least the portion of the first component or derivative thereof from the first separation stream comprises steam cracking the normal pentane from the first separation stream.

3. The method of claim 1, wherein the first component comprises isopentane, wherein the method further comprises converting at least a portion of the isopentane from the first separation stream to normal paraffins, and wherein steam cracking the normal paraffins from conversion of the second component and at least the portion of the first component or derivative thereof from the first separation stream comprises steam cracking the normal paraffins from conversion of at least a portion of the isopentane.

4. The method of claim 1, wherein the naphtha feedstock further comprises normal paraffins having at least six carbon atoms, and wherein separating the naphtha feedstock comprises separating the naphtha feedstock to produce the first separation stream including the first component and the second separation stream including the second component and the normal paraffins having at least six carbon atoms.

5. The method of claim 4, further comprising adsorbing the normal paraffins having at least six carbon atoms from the second separation stream to produce an extract stream and a raffinate stream, wherein the extract stream comprises the normal paraffins having at least six carbon atoms and the raffinate stream comprises the second component, and wherein steam cracking the normal paraffins from conversion of the second component and at least the portion of the first component or derivative thereof from the first separation stream further comprises steam cracking the normal paraffins having at least six carbon atoms from the extract stream.

6. The method of claim 1, wherein converting at least the portion of the second component from the second separation stream to normal paraffins comprises converting at least the portion of the second component from the second separation stream to produce a first conversion stream comprising normal paraffins having one or two carbon atoms, a second conversion stream comprising hydrocarbons having three or four carbon atoms, and a third conversion stream comprising conversion products having at least five carbon atoms.

7. The method of claim 6, wherein steam cracking normal paraffins from the conversion of the second component and at least the portion of the first component or derivative thereof from the first separation stream comprises steam cracking the normal paraffins having one or two carbon atoms from the first conversion stream.

8. The method of claim 6, wherein the second conversion stream comprises isobutane and at least one of propane and normal butane, wherein the method further comprises separating the propane and/or normal butane from the isobutane, and wherein steam cracking normal paraffins from conversion of the second component and at least the portion of the first component or derivative thereof from the first separation stream comprises steam cracking the propane and/or the normal butane from the second conversion stream.

9. The method of claim 8, further comprising converting at least a portion of the isobutane from the second conversion stream to normal paraffins, and wherein steam cracking the normal paraffins from conversion of the second component and at least the portion of the first component or derivative thereof from the first separation stream further comprises steam cracking the normal paraffins from conversion of at least the portion of the isobutane from the second conversion stream.

10. The method of claim 6, wherein the third conversion stream comprises conversion products having five carbon atoms and conversion products having at least six carbon atoms, and wherein the method further comprises separating the third conversion stream to produce a separated conversion stream comprising the conversion products having at least six carbon atoms and a recycle stream comprising conversion products having five carbon atoms.

11. The method of claim 10, wherein the first component and the recycle stream comprise isopentane and normal pentane, wherein the method further comprises combining the recycle stream and the first separation stream to form a combined pentane stream and separating the isopentane from the normal pentane in the combined pentane stream.

12. (canceled)

13. The method of claim 1, wherein reverse isomerizing at least the portion of the second component from the second separation stream produces normal paraffins and isoparaffins having from one to greater than five carbon atoms, and wherein the method further comprises separating normal paraffins and isoparaffins having at least six carbon atoms from normal paraffins and isoparaffins having five carbon atoms or less and again reverse isomerizing the normal paraffins and isoparaffins having at least six carbon atoms.

14. (canceled)

15. (canceled)

16. The method of claim 1, wherein steam cracking the normal paraffins produces ethylene, propylene, and steam-cracked hydrocarbons having at least five carbon atoms, and wherein the method further comprises recovering at least a portion of the steam-cracked hydrocarbons having at least five carbon atoms for converting to normal paraffins with at least the portion of the second component from the second separation stream.

17. The method of claim 1, wherein:
the converting at least the portion of the second component from the second separation stream to normal paraffins produces a first conversion stream comprising normal paraffins having one or two carbon atoms, a second conversion stream comprising hydrocarbons having three or four carbon atoms, and a third conversion stream comprising normal pentane, isopentane, and normal paraffins and isoparaffins having at least six carbon atoms;
separating the third conversion stream to produce a separated conversion stream comprising the normal paraffins and isoparaffins having at least six carbon atoms and a recycle stream comprising the normal pentane and isopentane; and
reverse isomerizing at least a portion of separated conversion stream.

18. (canceled)

19. A method of producing ethylene and propylene from naphtha feedstock in an apparatus for steam cracking the naphtha feedstock, the method comprising:
providing the naphtha feedstock comprising:
a first component consisting of hydrocarbons having less than or equal to five carbon atoms;
a second component consisting of at least one of an isoparaffin component having at least six carbon atoms, a naphthene component having at least six carbon atoms, or an aromatic component having at least six carbon atoms; and
normal paraffins having at least six carbon atoms;
separating the naphtha feedstock in a depentanizer unit to produce a first separation stream including the first component and a second separation stream including the second component and the normal paraffins having at least six carbon atoms;
adsorbing the normal paraffins having at least six carbon atoms from the second separation stream in a normal paraffin adsorption stage to produce an extract stream and a raffinate stream, wherein the extract stream comprises the normal paraffins having at least six carbon atoms and the raffinate stream comprises the second component;
converting by single stage reverse isomerization in the presence of a reverse isomerization catalyst at least a portion of the second component from the second separation stream in a conversion stage to produce a first conversion stream comprising normal paraffins having one or two carbon atoms, a second conversion stream comprising hydrocarbons having three or four carbon atoms, and a third conversion stream comprising conversion products having at least five carbon atoms; and
steam cracking the normal paraffins from at least one of;
(i) the first conversion stream;
(ii) the second conversion stream;
(iii) at least a portion of the first component or derivative thereof from the first separation stream; or
(iv) the normal paraffins having at least six carbon atoms from the extract stream;
in a steam cracking stage to produce ethylene and propylene.

20. (canceled)
